(19) United States
(12) Patent Application Publication
(10) Pub. No.: US 2007/0215148 A1
(43) Pub. Date: Sep. 20, 2007

(54) Secured Ventilated Breathing Device, in Particular for Hostile Medium

(75) Inventor: Swan Tuffery, Nimes (FR)

Correspondence Address: YOUNG & THOMPSON, 745 SOUTH 23RD STREET, 2ND FLOOR, ARLINGTON, VA, 22202, US

(73) Assignee: Delta Protection, Bagnols-Sur-Ceze (FR)

(21) Appl. No.: 11/578,234

(22) PCT Filed: Apr. 18, 2005
(86) PCT No.: PCT/FR05/00918

§ 371 Date: Oct. 12, 2006

(30) Foreign Application Priority Data
Apr. 27, 2004 (FR) 0404411

(51) Int. Cl. A61M 16/00

(52) U.S. Cl. 128/202.22

(57) ABSTRACT

A safe ventilated breathing apparatus to be worn by people in an ambient medium where there is an atmosphere at a pressure Pₐ that is substantially constant. The breathing apparatus includes a covering constituted by a skin of leakproof material, a fluid feed tube having one end suitable for being connected to a source of fluid at a given pressure Pₙ greater than Pₐ, and having its other end passing through the skin via a leaktight passage so as to plunge into the covering, vent elements made in the skin to put the inside of the covering into communication with the ambient medium, and elements for visually warning the person when the pressure of the fluid in the feed tube drops below a given threshold value Pₛ, less than Pₙ. The invention is particularly applicable to safe ventilated breathing apparatuses for the nuclear, bacteriological, etc., fields, hospital environments, etc.

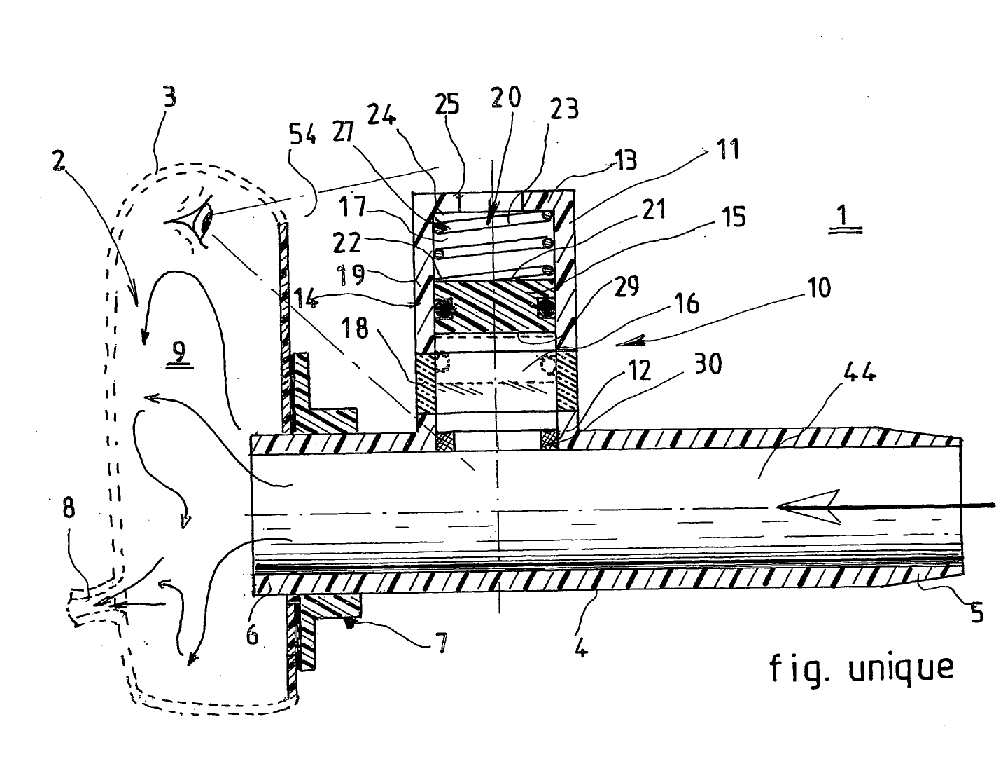

The present invention relates to safe ventilated breathing apparatuses, in particular for use in a hostile medium, finding a particularly advantageous application in confinement garments for protection purposes in nuclear, bacteriological, hospital medium, etc. surroundings, i.e. garments that satisfy the safety standards that are applicable in particular to the nuclear, bacteriological, chemical, biological, etc. fields, and it even relates to garment type equipments for reducing thermal stress, for example.

It is known that there is a need to protect people working in a hostile medium as defined above, for example.

One of the means used for protecting such people is to have them wear a breathing apparatus which covers them either in full, if the apparatus is of the “total” type, or in part only, if the apparatus is of the “helmet”, “hood”, or similar type that covers the upper portion of the body to a greater or lesser extent.

In order to ensure that harmful substances cannot penetrate into the apparatus and in order to allow the person wearing such apparatus to breathe normally, the apparatus further includes means that enable its inside to be ventilated, while maintaining small positive pressure therein, by using a fluid under pressure such as clean industrial air or the like.

For this purpose, a breathing apparatus is constituted by a skin that defines a covering in which the person for protection can be enclosed either in full or in part, as mentioned above, together with a feed tube for feeding fluid under pressure, having one end adapted to be connected to a source of said fluid situated in a clean location, and having its other end plunging into the covering by passing through the skin via a leaktight passage, using a duct of length that is sufficient to allow the person wearing such an apparatus to reach all of the targets for that person in the medium in which that person is to move.

In addition, when the person is wearing such apparatus, the person is generally in a medium that is hostile and is very busy with the tasks that are to be performed. It is therefore necessary for that person to be able to breathe normally in complete safety. For this purpose, it is absolutely necessary to give warning as quickly as possible when there is a risk of a breakdown occurring in the fluid feed.

It is therefore prudent to provide the breathing apparatus with a warning device to enable the person wearing it to go to a safe place on being warned of a risk of such a breakdown, and while sufficient fluid remains to protect the person and allow breathing. Such devices are described in DE 197 00 229 and U.S. Pat. No. 4,455,683, for example.

Thus, an object of the present invention is to provide a breathing apparatus that is ventilated by means of a given fluid, that tends as well as possible to reach the objectives defined below.

More precisely, the present invention provides a safe ventilated breathing apparatus for a person, when said apparatus is for use in an ambient medium where there is an atmosphere at a pressure Pₐ that is substantially constant, and as defined in accompanying claim 1.

Other characteristics and advantages of the invention appear from the following description with reference to the accompanying drawing by way of illustrative but non-limiting example, in which:
the sole figure is a longitudinal section view of a preferred embodiment of a safe ventilated breathing apparatus of the invention.

Firstly it is specified that the figure shows essentially only one embodiment of the subject matter of the invention, but that other embodiments can also exist that satisfy the definition of the invention.

That specified, the sole figure shows an embodiment of a safe ventilated breathing apparatus of the invention that finds a particularly advantageous application, as mentioned above, in the field of providing confinement for protection purposes in nuclear, bacteriological, hospital medium, etc., surroundings, it being specified that such a safe ventilated breathing apparatus is for wearing by a person who needs to move and work in a surrounding medium in which there is an atmosphere at a pressure Pₐ that is substantially constant, for example atmospheric pressure.

Such a breathing apparatus comprises a covering 2 constituted by a skin 3 of leakproof material, for example of polyvinylchloride (PVC). It also comprises a feed tube 4 for feeding a given fluid such as industrial air or the like, having a first end 5 suitable for connection to a source of the given fluid (not shown) suitable for delivering said given fluid at a pressure Pₙ greater than the pressure Pₐ, and having its other end 6 passing through the skin 3 via a leaktight passage 7 so as to extend inside the covering 2.

It also comprises vent means 8 formed in the skin 3 to put the inside 9 of the covering into communication with the ambient medium 1 outside it. These means may be of various forms and are well known in themselves. They are thus not described in fuller detail herein, solely for the purpose of simplifying the present description.

The breathing apparatus also comprises means 10 for visually warning the person wearing the apparatus when the pressure of the fluid in the feed tube 4 drops below a given threshold value Pₛ that is less than Pₙ.

In a preferred embodiment, these means 10 for visually warning the person when the pressure of the fluid in the feed tube falls below a given threshold value Pₛ less than Pₙ, are constituted by a duct 11 having a first end 12 in fluid connection with the feed tube 4 and a second end 13 connected to the ambient medium 1, the duct 11 including, between its first and second ends 12 and 13, at least one portion 14 having an inside that is in the form of a cylinder, advantageously a circular cylinder.

A piston 15 is also provided that is mounted to slide in leaktight manner inside said cylindrical duct portion 14 in order to define therein first and second chambers 16 and 17 of volumes that are variable in mutually opposite directions, the first chamber 16 being in communication with the inside 44 of the feed tube 4, and the second chamber 17 being in communication with the ambient medium 1.

These means 10 also include a transparent leakproof window 18 formed in the wall 19 of the cylindrical inside duct portion 14 and situated on the path followed by the piston 15 during sliding, and means 20 for applying a resilient force against the face 21 of the piston 15 that is adjacent to the second chamber 17, the magnitude of this resilient force varying inversely in proportion to the variation in the volume of the second chamber 17. In addition, this resilient force is determined in such a manner that the piston 15 covers at least part of said transparent window 18 when the pressure in the feed tube 4 is less than Pₛ, and such that the piston lies outside said window when the pressure in the feed tube 4 is not less than Pₛ.

Preferably, the duct 11 is mounted in association with the feed tube 4 in such a manner that the transparent window 18 is situated in the field of view 54 of the person wearing the breathing apparatus, for the reason explained below in the description of the operation of the safe breathing apparatus of the invention.

It is also advantageous for the transparent window 18 to be constituted by an annular portion of transparent material in the side wall of the first chamber 16.

In addition, in an embodiment that is preferred from industrial and economic points of view, the means 20 for applying the resilient force are constituted, as shown in the figure, by a helical type compression spring 27 situated in the second chamber 17. One of the ends 22 of the spring 27 is mounted in contact with the face 21 of the piston 15 that is adjacent to the second chamber 17, and its other end 23 is mounted in contact with an abutment 24 formed in said second chamber 17. By way of example, the abutment 24 is constituted by a collar 25 secured to the wall 19 of the cylindrical portion of the duct, said collar 25 projecting towards the inside of the chamber 17.

For the purpose explained below, it is also advantageous for the wall of the piston 15 to be of a bright color.

Finally, to ensure that the piston cannot escape from the first chamber 16, e.g. coming to obstruct the feed tube 4, it is preferable for the visual warning means 10 to include means 30 for limiting the sliding of the piston into the first chamber 16, e.g. a stud or the like situated at the end 12 of the cylindrical duct portion 14.

A preferred embodiment of the safe ventilated breathing apparatus of the invention is described above. However it is naturally possible to make such breathing apparatus in other embodiments. For example, as described and shown the duct is of constant section over its entire length. However it is possible to make a cylindrical portion as two portions having two different sections that are joined together by a step, and a matching piston likewise comprising two different sections joined together by a corresponding step so as to obtain, for example, a face 29 of the piston that is subjected to the pressure of the fluid flowing in the feed tube that is smaller than the face 21 against which the spring 27 and the pressure Pₐ act, with the step then constituting the means 30 for limiting sliding of the piston into the first chamber 16 and for preventing it from partially or completely obstructing the feed tube 4.

The converse configuration could also be envisaged, i.e. a face 29 that is larger than the face 21, with the means 30 for limiting sliding of the piston then being constituted as shown in the figure.

Similarly, a helical spring is shown and given as an embodiment that is preferred for industrial and economic purposes. However other embodiments are possible, for example a pneumatic spring or the like.

Similarly, it is also possible for the cylindrical duct portion 14 that is shown as being connected directly to the feed tube 4 and to the ambient medium 1, to be connected indirectly to said feed tube 4 and to said ambient medium 1, e.g. by means of long pipes or the like. This applies for example when it is necessary to place the leaktight passage at a location that is more favorable for making the breathing apparatus but that lies outside the field of view of the person wearing the apparatus. Under such circumstances, the cylindrical duct portion 14 should be connected to the feed tube 4 by means of an auxiliary pipe of length enabling it to be positioned together with its transparent window 18 in the field of view.

The above-described breathing apparatus operates as follows:

It is assumed initially that a person wearing a breathing apparatus of the invention as described above needs to move in a hostile medium. For this purpose, the end 5 of the feed tube 4 is connected to a source of fluid, e.g. industrial air, at a pressure Pₙ greater than the pressure Pₐ of the ambient medium. The inside 9 of the covering 2 is thus put to a high pressure so as to allow the person to breathe normally and so as to allow the covering to be ventilated normally, the vent means 8 enabling a relatively constant pressure to be maintained inside the covering 2 and the air expelled through the vent means ensuring that dangerous substances do not penetrate into the inside of the covering.

When the pressure in the feed tube, and thus also in the first chamber 16, is normal and equal to Pₙ, then a force acts on the face 29 of the piston that is equal to Pₙ×S, where S is the area of said face 29. Under the effect of this thrust force, the piston is pushed towards the end 13 of the cylindrical duct portion 14, compressing the spring 27 until the thrust force exerted on the face 29 and the forces exerted by the spring and the ambient medium on the face 21 of the piston come into equilibrium, thus holding the piston 15 in a position away from the transparent window 18. This position is drawn in continuous lines in the sole figure.

In contrast, if for any reason whatsoever, the pressure inside the feed tube drops, then the thrust force exerted on the face 29 of the piston 15 decreases and the spring 27 pushes against the piston so as to tend to reduce the volume of the first chamber 16.

If the pressure in the feed tube drops to a value below the threshold pressure Pₛ, the piston is pushed back until it is to be found at least in part in register with the transparent window 18, overlying it at least in part, as shown in dashed lines in the figure.

Since, by structural definition, the transparent window 18 lies in the field of view 54 of the person wearing the breathing apparatus, that person is immediately aware that the pressure of the fluid inside the feed tube is no longer sufficient to guarantee safety, and that it is therefore necessary to take appropriate action to leave the hostile medium and preserve safety.

Having a bright color for the piston serves to further attract the attention of the person wearing the breathing apparatus.

From the above description, it can easily be understood that the ventilated breathing apparatus of the invention is very simple to make, inexpensive, and very reliable in safety terms since, in addition, it does not include any electrical or similar elements that might harm the safety of people needing to wear such breathing apparatuses.

1. A safe ventilated breathing apparatus for a person, said apparatus being suitable for being used in an ambient medium (1) in which there exists an atmosphere at a pressure Pₐ that is substantially constant, said apparatus comprising:
a covering (2) made of a skin (3) of leakproof material;
a feed tube (4) for feeding a fluid, one end (5) of the tube being suitable for being connected to a source of said fluid at a given pressure Pₙ greater than the pressure Pₐ, and the other end (6) thereof passing through the skin via a leaktight passage (7) so as to plunge inside the covering; and
vent means (8) made in the skin to put the inside (9) of the covering into communication with the ambient medium (1);
the apparatus being characterized by the fact that it further comprises means (10) for visually warning said person when the pressure of the fluid in said feed tube (4) drops below a given threshold pressure Pₛ less than Pₙ, said means being constituted by a duct (11) having a first end (12) in fluid connection with the feed tube (4) and a second end (13) connected to the ambient medium (1), said duct (11) including, between its first and second ends (12, 13), a portion (14) having an inside that is cylindrical in shape, a piston (15) mounted to slide in leaktight manner in the cylindrical duct portion (14) and defining in said duct portion first and second chambers (16, 17) presenting volumes that are variable in mutually opposite directions, the first chamber (16) being in communication with the inside (44) of the feed tube (4) and the second chamber (17) being in communication with the ambient medium (1), a transparent leakproof window (18) formed in the wall (19) of the cylindrical duct portion (14) and situated on the path followed by the piston (15) during sliding, and means (20) for applying a resilient force against the face (21) of the piston (15) that is adjacent to the second chamber (17), the magnitude of said resilient force varying inversely in proportion to the variation in the volume of said second chamber (17), said resilient force being determined so that the piston (15) overlies at least part of the transparent window (18) when the pressure in the feed tube (4) is less than Pₛ, and so that the piston (15) lies outside said transparent window (18) when the pressure in the feed tube (4) is not less than Pₛ.

2. A safe ventilated breathing apparatus according to claim 1, characterized by the fact that the duct (11) is mounted in association with the feed tube (4) in such a manner that the transparent window (18) lies in the field of view (54) of the person when wearing the apparatus.

3. A safe ventilated breathing apparatus according to claim 1, characterized by the fact that the transparent window (18) is constituted by an annular portion of the side wall of the first chamber, which portion is made of transparent material.

4. A safe ventilated breathing apparatus according to claim 1, characterized by the fact that the means (20) for applying the resilient force are constituted by a compression spring (27) situated in the second chamber (17), having one end (22) mounted in contact with the face (21) of the piston (15) that is adjacent to said second chamber (17), and having its other end (23) mounted in contact with an abutment (24) formed in said second chamber (17).

5. A safe ventilated breathing apparatus according to claim 4, characterized by the fact that said abutment (24) is constituted by a collar (25) secured to the wall (19) of the cylindrical duct portion, said collar (25) projecting towards the inside of said second chamber (17).

6. A safe ventilated breathing apparatus according to claim 1, characterized by the fact that the wall of said piston (15) is of a bright color.

7. A ventilated breathing apparatus according to claim 1, characterized by the fact that the means (10) for visually warning said person further comprise means (30) for limiting sliding of the piston in the first chamber.

8. A safe ventilated breathing apparatus according to claim 2, characterized by the fact that the transparent window (18) is constituted by an annular portion of the side wall of the first chamber, which portion is made of transparent material.

9. A safe ventilated breathing apparatus according to claim 2, characterized by the fact that the means (20) for applying the resilient force are constituted by a compression spring (27) situated in the second chamber (17), having one end (22) mounted in contact with the face (21) of the piston (15) that is adjacent to said second chamber (17), and having its other end (23) mounted in contact with an abutment (24) formed in said second chamber (17).

10. A safe ventilated breathing apparatus according to claim 3, characterized by the fact that the means (20) for applying the resilient force are constituted by a compression spring (27) situated in the second chamber (17), having one end (22) mounted in contact with the face (21) of the piston (15) that is adjacent to said second chamber (17), and having its other end (23) mounted in contact with an abutment (24) formed in said second chamber (17).
